(19) United States
(12) Patent Application Publication
(10) Pub. No.: US 2020/0039901 A1
(43) Pub. Date: Feb. 6, 2020

(54) METHOD FOR DECOMMISIONING AND REGENERATING A REACTOR FOR THE OXIDATIVE DEHYDROGENATION OF N-BUTENES

(71) Applicants: BASF SE, Ludwigshafen am Rhein (DE); Linde AG, München (DE)

(72) Inventors: Jan UNGELENK, Ludwigshafen am Rhein (DE); Oliver HAMMEN, Ludwigshafen am Rhein (DE); Christian WALSDORFF, Ludwigshafen am Rhein (DE); Rainer ECKRICH, Ludwigshafen am Rhein (DE); Heinz BOELT, München (DE); Christine TOEGEL, München (DE); Ulrike WENNING, München (DE); Hendrik REYNEKE, München (DE); Anton WELLENHOFER, München (DE)

(21) Appl. No.: 16/498,693

(22) PCT Filed: Mar. 26, 2018
(86) PCT No.: PCT/EP2018/057632

§ 371 Date: Sep. 27, 2019

(30) Foreign Application Priority Data
Mar. 31, 2017 (EP) 17164313.3

(51) Int. Cl. C07C 11/167; C07C 7/04; C07C 7/11; C07C 5/333

(52) U.S. Cl. CPC C07C 11/167; C07C 5/333; C07C 7/11; C07C 7/04

(57) ABSTRACT

The invention relates to a process for preparing butadiene from n-butenes in n reactors R1 to Rn operated in parallel, wherein the process in the production phase of a reactor Rm in the n reactors comprises the steps:
A) provision of a feed gas stream a1m comprising n-butenes;
B) feeding of the feed gas stream a1m comprising n-butenes, an oxygen-comprising gas stream a2m and a substream d2m of an oxygen-comprising total recycle gas stream d2 into the oxidative dehydrogenation zone of the reactor and oxidative dehydrogenation of n-butenes to butadiene, giving a product gas substream bm comprising butadiene;
C) combination of the product gas substream bm with further product gas substreams to form a total product gas stream b and cooling and compression of the total product gas stream b and condensation of at least part of the high-boiling secondary components, giving at least one aqueous condensate stream c1 and a gas stream c2 comprising butadiene;
D) feeding of the gas stream c2 into an absorption zone and separation of incondensable and low-boiling gas constituents as gas stream d from the gas stream c2 by absorption of the C₄-hydrocarbons in an absorption medium, giving an absorption medium stream d1 loaded with C₄-hydrocarbons and a recycle gas stream d2, and recirculation of a substream d2m of the total recycle gas stream d2 into the reactor Rm,
and during the regeneration phase of the reactor Rm further reactors are in the production phase and the regeneration phase of the reactor Rm comprises the steps in the order i) to v):
i) reduction of the feed gas stream a1m comprising n-butenes and of the oxygen-comprising gas stream a2m and the feeding of an inert gas stream a4m into the reactor Rm;
ii) further reduction of the feed gas stream a1m comprising n-butenes down to 0, reduction of the recycle gas substream d2m and increase of the inert gas stream a4m until the oxygen content in the reactor Rm is from 2 to 3% by volume;
iii) regeneration of the catalyst by burning off carbon-comprising deposits at an oxygen content of from 2 to 3% by volume;
iv) reduction of the inert gas stream a4m and increase of the recycle gas substream d2m until the oxygen content in the reactor Rm is from 4 to 10% by volume;
v) reoxidation of the catalyst at an oxygen content of from 4 to 10% by volume.

The invention relates to a process for running down and regenerating a reactor for preparing 1,3-butadiene from n-butenes by oxidative dehydrogenation (ODH).

Butadiene is an important basic chemical and is used, for example, for producing synthetic rubbers (butadiene homopolymers, styrene butadiene rubber or nitrile rubber) or for producing thermoplastic terpolymers (acrylonitrile butadiene-styrene copolymers). Butadiene is also converted into sulfolane, chloroprene and 1,4-hexamethylendiamine (via 1,4-dichlorobutene and adiponitrile). Furthermore, vinylcyclohexene can be produced by dimerization of butadiene and this can be dehydrogenated to styrene.

Butadiene can be prepared by thermal dissociation (steam cracking) of saturated hydrocarbons, with naphtha usually being employed as raw material. The steam cracking of naphtha gives a hydrocarbon mixture of methane, ethane, ethene, acetylene, propane, propene, propyne, allene, butanes, butenes, butadiene, butines, methylallene, C₅-hydrocarbons and higher hydrocarbons.

Butadiene can also be obtained by oxidative dehydrogenation of n-butenes (1-butene and/or 2-butane). As feed gas for the oxidative dehydrogenation (oxydehydrogenation, ODH) of n-butenes to butadiene, it is possible to utilize any mixture comprising n-butenes. For example, it is possible to use a fraction which comprises n-butenes (1-butene and/or 2-butene) as main constituent and has been obtained from the C₄ fraction from a naphtha cracker by separating off butadiene and isobutene. Furthermore, gas mixtures which comprise 1-butane, cis-2-butene, trans-2-butene or mixtures thereof and have been obtained by dimerization of ethylene can also be used as feed gas. Gas mixtures which comprise n-butenes and have been obtained by fluid catalytic cracking, (FCC) can also be used as feed gas.

The reaction of the gas streams comprising butenes is usually carried out industrially in shell-and-tube reactors which are operated in a salt bath as heat transfer medium. The product gas stream is cooled downstream of the reactor by direct contact with a coolant in a quenching stage and is subsequently compressed. The C₄ components are then absorbed in an organic solvent in an absorption column. Inert gases, low boilers, CO, CO₂ and others leave the column at the top. Part of this overhead stream is fed as recycle gas to the ODH reactor. Hydrocarbons and oxygen can produce an explosive atmosphere. The concentration of combustible gas constituents (mostly hydrocarbons and CO) can be below the lower explosive limit (LEL) or above the upper explosive limit (UEL) in order to avoid ignitable mixtures. Below the lower explosive limit, the oxygen concentration can be selected freely without an explosive gas mixture being able to form. However, the concentration of feed gas is then low, which is economically disadvantageous. For this reason, a reaction using a reaction gas mixture above the upper explosive limit is preferred. Here, whether an explosion can occur depends on the oxygen concentration. Below a particular oxygen concentration, the LOC (limiting oxygen concentration), the concentration of combustible gas constituents can be selected freely without an explosive gas mixture being able to form. Both LEL, UEL and LOC are temperature- and pressure-dependent.

In the oxidative dehydrogenation of n-butenes to 1,3-butadiene, it is possible for precursors of carbonaceous material, for example styrene, anthraquinone and fluorenone, to be formed and these can ultimately lead to carbonization and deactivation of the multimetal oxide catalyst. The pressure drop over the catalyst bed can increase as a result of the formation of carbon-comprising deposits. It is possible for carbon deposited on the multimetal oxide catalyst to be burned off by means of an oxygen-comprising gas at regular intervals in order to effect regeneration and restore the activity of the catalyst.

DE 734026 describes the regeneration of dehydrogenation catalysts based on aluminum oxide which comprise 5% of chromium oxide and are used, in particular, in a process for the dehydrogenation of butane. The regeneration is carried out at particular time intervals under the action of an oxygen-comprising gas. In order to keep the regeneration time short, preferably only part of the deposited carbonaceous material is burned off.

JP 60-058928 describes the regeneration of a multimetal oxide catalyst comprising at least molybdenum, bismuth, iron, cobalt and antimony for the oxidative dehydrogenation of n-butenes to 1,3-butadiene by means of an oxygen-comprising regeneration gas mixture at a temperature of from 300 to 700° C., preferably 350 to 650° C., and an oxygen concentration of from 0.1 to 5%. Air diluted with suitable inert gases such as nitrogen, steam or carbon dioxide is fed in as oxygen-comprising gas mixture.

WO 2005/047226 describes the regeneration of a multimetal oxide catalyst for the partial oxidation of acrolein to acrylic acid, which comprises at least molybdenum and vanadium, by passing an oxygen-comprising gas mixture over it at a temperature of from 200 to 450° C. As oxygen-comprising regeneration gas mixture, preference is given to using lean air comprising from 3 to 10% by volume of oxygen. Apart from oxygen and nitrogen, the gas mixture can comprise steam.

An inexpensive oxygen-comprising regeneration gas mixture is air. Thus, US 2007/0142689, states that dehydrogenation catalysts known from the prior art can be regenerated in a simple manner after a prolonged period of operation by passing preferably air, optionally diluted with N₂ or steam, over them at elevated temperatures (>300° C.).

The oxygen content of air is, at about 20.95% by volume, comparably high. Two important reasons stand in the way of a regeneration gas having a relatively high oxygen content: (1) In a shell-and-tube reactor, the amount and local distribution of the carbonaceous material cannot be predicted precisely. In an unfavorable case, a reaction tube is so greatly carbonized that virtually no flow through it occurs any longer. Heat removal is then greatly hindered and is determined by the oxygen content of the regeneration gas mixture. If the oxygen content is high, can large local temperature increases in the reaction tube can occur, which can lead to destruction of the catalyst or even of the reaction tube or of the entire reactor. (2) Precursors of carbonaceous material and carbon-comprising deposits can be present not only on the catalyst surface but also within the catalyst, e.g. In the pores. If a regeneration gas having a relatively high oxygen content which is significantly higher than the oxygen content during the operating phase is used for burning off the precursors of carbonaceous material and carbon-comprising deposits, rapid burning in which the carbon-comprising deposits are converted quickly into carbon oxides can occur. The associated rapid increase in volume due to gas evolution can irreversibly damage the catalyst by spalling of the active composition.

An oxygen-comprising regeneration gas mixture having an oxygen content lower than that of air, or a lower oxygen content than in the operating phase, can be produced by diluting air with inert gases, e.g. as described in US 2007/0142689 by dilution with nitrogen or steam. However, a high steam content in the regeneration gas mixture can damage the catalyst, e.g. in the case of Mo-comprising catalysts by formation and discharge of volatile molybdenum compounds. Inert gases; such as nitrogen, argon, neon, helium, carbon dioxide or mixture of these; are expensive. Furthermore, large and expensive vessels are necessary to keep a sufficient amount of inert gas.

In the abovementioned documents, nothing is said about how a regeneration gas mixture can be provided inexpensively and reliably. Furthermore, the above documents say nothing about how a switch from a production step to a regeneration step and vice versa is to be effected without there being a risk of an explosive atmosphere being produced in the reactor or in other regions of the plant.

A likewise advantageous gas mixture having a lower oxygen content than air is the recycle gas which is obtained by separating the incondensable or low-boiling gas constituents from the product gas mixture from the oxydehydrogenation and is recirculated to the oxydehydrogenation zone. However, in a typical operating state, the recycle gas comprises 7-9% by volume of oxygen, as a result of which the risk of high local temperature peaks is still very high.

A recycle gas of this type is obtained in the ODH process described in WO2015/007839, in which n-butenes are reacted with an oxygen-comprising gas, preferably air, to form butadiene. WO2015/007839 is concerned with the problem of the oxygen contents of recycle gas obtained in the process described, which are still relatively high for regeneration operation, and proposes the following solution: Alternating operating and regeneration phases. At the end of each production step, the introduction of air into the reactor is throttled back or shut off entirely, but oxydehydrogenation operation is continued until the oxygen concentration in the recycle gas has dropped to 5% by volume. The introduction of the gas stream comprising the n-butenes and the introduction of the oxygen-comprising gas are then stopped. The subsequent regeneration step is carried out using the resulting recycle gas having an oxygen content of 5% by volume.

A significant element of the process described in WO2015/007839 is thus that the oxygen content of the recycle gas in the regeneration phase is significantly below the oxygen content of the recycle gas in the production phase. All the above-mentioned documents proceed from a single reactor in which the production phase and the regeneration phase are carried out alternately. No information is given as to how the energy for the regeneration step is provided. Furthermore, nothing is said about possible parallel operation of a plurality of reactors which can independently be in the production phase or regeneration phase.

WO2015/007841 describes an ODH process in which two shell-and-tube reactors operated in parallel and independently of one another are present in a joint heat transfer medium. Each production line comprises a shell-and-tube reactor, a quenching section and a compressor section, after which the two compressed product gas streams are fed together into a joint absorption column. There, an oxygen-comprising recycle gas is obtained by removal of the incondensable or low-boiling gas constituents of the combined product gas from the oxydehydrogenation and a substream is recirculated to each of the two reactors in the production phase.

In the process described in WO2015/007841, the transition of one of the two reactors from the production phase to the regeneration phase is configured so that the oxygen-comprising gas stream, preferably air, is firstly shut off while the oxydehydrogenation continues to run until the concentration of oxygen in the product gas stream from this reactor has dropped to below a prescribed value. Then, firstly the substream of the recycle gas stream and then the butene-comprising feed stream for this reactor are shut off, whereupon the regeneration mode is started by discharging the compressed product gas stream from the reactor upstream of the joint absorption column. A stream comprising inert gas is then fed into the regenerating reactor and a substream of the stream leaving the reactor is subsequently compressed and recirculated as oxygen-comprising regeneration gas to the reactor under consideration. During this time, the second production line remains in the production mode.

However, the procedure described in WO2015/007841 is associated with a number of disadvantages. Thus, the two production lines are each supplied with a substream of the joint recycle gas only during the production phase but not during the regeneration phase. For the regeneration phase, a regeneration gas for each line has to be separately produced, preheated, compressed and recirculated. For this reason, at least two compressor sections have to be kept available per production line—one for the production phase and at least one for the regeneration phase. The regeneration gas is produced by interrupting the oxygen-comprising feed streams, preferably air, and a substream of the recycle gas from the production phase and instead feeding in an inert gas, while oxydehydrogenation operation is firstly continued in order to decrease the oxygen content of the product gas stream before the n-butene-comprising feed stream is finally also interrupted. Here, the oxydehydrogenation reaction proceeds increasingly less selectively, so that increased amounts of precursors of carbonaceous material and carbonaceous material are formed during the transition from production phase to the regeneration phase. The risk of individual reaction tubes carbonizing to a greater extent thus also increases, as a result of which local overheating of the tubes concerned can occur in the subsequent regeneration process and can lead to damage to the catalyst in the tube concerned and in the worst case to damage to the reactor. In addition, the precursors of carbonaceous material which are formed in increased amounts are recirculated to the reactor during the regeneration phase since a substream of the reactor output stream is used as regeneration gas. The efficiency of the regeneration process is thus reduced. A further disadvantage concerns the work-up which is carried out jointly for the two production lines and is subject to large fluctuations both in the total flow and in the C4 loading. The work-up section alternates between 100% load operation when both lines are in the production phase and 50% minimum load operation when one line is in the production phase and the other line is in the regeneration phase.

For the regeneration of a catalyst by means of an oxygen-comprising regeneration gas, the requirements which the gas atmosphere has to meet, in particular in respect of the oxygen content, are thus different for the production phase and the regeneration phase. The transition from the production phase to the regeneration phase, known as the running-down phase, presents a particular challenge. The processes described in WO2015/007839 and WO2015/007841 have disadvantages such as Increased formation of precursors of carbonaceous material and carbon-comprising compounds. The oxygen content must not be too high either in the production phase or in the regeneration phase so that no ignitable mixtures are formed. At the start of the regeneration phase, the oxygen content must not be too high in order to avoid excessively rapid burning-off of carbon-comprising compounds and local overheating of possibly greatly carbonized reaction tubes, which can lead to destruction of the catalyst and of the reaction tubes.

In the production phase, the oxygen content must not be too low in order to avoid premature or excessive carbonization of the catalyst. In the transition phase from production phase to regeneration phase, a change in the oxygen content based on the n-butene-comprising feed gas should occur in such a way that neither does the carbonization tendency increase nor does premature or excessively rapid burning-off occur, nor is an ignitable mixture produced.

It is an object of the invention to provide a safe and economical process for running down a reactor for the oxidative dehydrogenation of n-butenes to butadiene.

The object is achieved by a process for preparing butadiene from n-butenes in n reactors R1 to Rn operated in parallel, with each of the plurality of reactors operated in parallel going through a production phase and a regeneration phase, wherein the process in the production phase of a reactor Rm in the n reactors comprises the steps:
A) provision of a feed gas stream a1m comprising n-butenes;
B) feeding of the feed gas stream a1m comprising n-butenes, an oxygen-comprising gas stream a2m and a substream d2m of an oxygen-comprising total recycle gas stream d2 into the oxidative dehydrogenation zone of the reactor and oxidative dehydrogenation of n-butenes to butadiene, giving a product gas substream bm comprising butadiene, unreacted n-butenes, water vapor, oxygen, low-boiling hydrocarbons, high-boiling secondary components, possibly carbon oxides and possibly inert gases;
C) combination of the product gas substream bm with further product gas substreams to form a total product gas stream b and cooling and compression of the total product gas stream b and condensation of at least part of the high-boiling secondary components, giving at least one aqueous condensate stream c1 and a gas stream c2 comprising butadiene, n-butenes, water vapor, oxygen, low-boiling hydrocarbons, possibly carbon oxides and possibly inert gases;
D) feeding of the gas stream c2 into an absorption zone and separation of incondensable and low-boiling gas constituents comprising oxygen, low-boiling hydrocarbons, possibly carbon oxides and possibly inert gases as gas stream d from the gas stream c2 by absorption of the C₄-hydrocarbons comprising butadiene and n-butenes in an absorption medium, giving an absorption medium stream d1 loaded with C₄-hydrocarbons and a recycle gas stream d2, and recirculation, optionally after a purge gas stream p has been separated off, of a substream d2m of the total recycle gas stream d2 into the reactor Rm,
wherein during the regeneration phase of the reactor Rm further reactors are in the production phase and the regeneration phase of the reactor Rm comprises the steps in the order i) to v):
i) reduction of the feed gas stream a1m comprising n-butenes and of the oxygen-comprising gas stream a2m and feeding of an inert gas stream a4m into the reactor Rm, with the total gas flow through the reactor Rm corresponding to from 55 to 75%, preferably from 60 to 70%, of the total gas flow through the reactor Rm during the production phase;
ii) further reduction of the feed gas stream a1m comprising n-butenes down to 0, reduction of the recycle gas substream d2m and increase of the inert gas stream a4m until the oxygen content in the reactor Rm is from 2 to 3% by volume, with the total gas flow through the reactor Rm corresponding to from 55 to 75%, preferably from 60 to 70% of the total gas flow through the reactor Rm during the production phase;
iii) regeneration of the catalyst by burning off carbon-comprising deposits at an oxygen content of from 2 to 3% by volume;
iv) reduction of the inert gas stream a4m and increase of the recycle gas substream d2m until the oxygen content in the reactor Rm is from 4 to 10% by volume, with the total gas flow through the reactor Rm corresponding to from 60 to 120% of the total gas flow during the production phase;
v) reoxidation of the catalyst at an oxygen content of from 4 to 10% by volume.

An advantage of the process of the invention is that the reoxidation of the catalyst in step iv) can be carried out using the recycle gas substream d2m generated by the reactors in the production state. This has an oxygen content of generally from 4 to 10% by volume, preferably from 6.5 to 9% by volume, which is favorable for the reoxidation of the catalyst. Additional inert gas is required only in step i) to iii), where the gas substream d2m has to be diluted to a lesser extent with inert gas in order to set the oxygen content of from 2 to 3% by volume which is favorable for the burning-off phase iii).

The oxygen:n-butenes ratio during step i) is preferably from 0.7 to 1.0 times the ratio in the production phase.

Furthermore, the oxygen content in the reactor Rm is preferably reduced to from 3 to 7% by volume during step i).

Furthermore, the gas stream a1m comprising n-butenes is preferably reduced during step i) to from 25 to 50% of the gas flow during the production phase.

Furthermore, preference is given to the gas stream a1m comprising n-butenes being reduced during step ii) to from 12.5 to 25% of the gas flow during the production phase, and the gas substream d2m being reduced correspondingly so that the oxygen:n-butenes ratio is from 0.7 to 1.0 times the ratio during the production phase, and the gas stream a1m comprising n-butenes subsequently being reduced to 0.

The reoxidation of the catalyst is carried out at an oxygen content of from 4 to 10% by volume, preferably from 6.5 to 9% by volume.

Step iii) is preferably carried out over a period of from 5 to 60 minutes. Step v) is preferably carried out over a period of from 12 to 96 hours.

The ratio of oxygen to hydrocarbons in the production phase is, in the case of a content of n-butenes of from 50 to 100% by volume in the feed gas stream a1, generally from 1:1 to 1.65:1, preferably from 1.3:1 to 1.65:1, particularly preferably from 1.4:1 to 1.65:1.

In general, the pressure in the reactor Rm during the production phase and regeneration phase is from 1 to 5 bar.

In one embodiment of the invention, the oxygen-comprising gas stream a2m is reduced to 0 in step i) or in step ii), preferably in step i). In a further embodiment of the invention, the inert gas stream a4m is reduced to 0 in step iv).

Particularly preferred variants of the process of the invention are explained in more detail below.

FIG. 1 schematically shows a plant for carrying out an embodiment of the process of the invention having a first shell-and-tube reactor R¹, a second shell-and-tube reactor R² and optionally further shell-and-tube reactors Rⁿ, which have a joint quenching section Q, compressor section K and absorption column A. Here,

R¹, R², Rⁿ are n reactors operated in parallel,
a1¹, a2¹, a3¹, a1², a2², a3², a1ⁿ, a2ⁿ, a3ⁿ are the streams a1, a2, a3 assigned to the individual reactors,
d2ₜₒₜₐₗ is the total recycle gas stream,
d2¹, d2², d2ⁿ are the recycle gas substreams assigned to the individual reactors,
b¹, b², bⁿ are the product gas substreams assigned to the individual reactors,
b is the combined butadiene-comprising C₄ product gas stream,
Q is a quenching stage,
K is a compression stage,
c1 is an aqueous condensate stream,
c2 is the butadiene-comprising C₄ product gas stream,
A is the absorption stage,
d1 is the butadiene-comprising C₄ product gas stream,
d is the total recycle gas stream before a purge stream has been separated off,
p is the purge stream and
q¹, q², qⁿ are the output streams assigned to the individual reactors.

In the production mode (production phase) of a reactor Rm, a feed gas stream a1m comprising the n-butenes is mixed with an oxygen-comprising gas stream a2m and passed as mixed gas stream over the heterogeneous particulate multimetal oxide catalyst with which the contact tubes of the shell-and-tube reactors Rm have been charged. The heat transfer medium takes up the heat of reaction liberated minus the quantity of heat consumed for heating the mixed gas stream to reaction temperature in the production mode by indirect heat exchange and transfers all or part of this to a secondary heat transfer medium in an external cooler. A major part of the high-boiling secondary components and of the water are separated off from the combined product gas substreams bm from the individual reactors Rm in a quench Q by contacting with a coolant to give a quenching gas stream which is compressed in a compression stage K and fed as compressed stream c2 to an absorption column. Here, a first reactor R1, a second reactor R2 and optionally further reactors R3 to Rn have a joint quench Q, a joint compressor K and a joint absorption column A from which an overhead stream d2 is taken off and is recirculated partly as first recycle gas stream d21 to the first shell-and-tube reactor R1, partly as second recycle gas stream d22 to the second shell-and-tube reactor R2 and as further recycle gas streams d23 to d2n to further optional shell-and-tube reactors R3 to Rn. The remainder of the stream d2 is discharged as purge gas stream p. A substream of the stream d2 can optionally be recirculated as compensation stream d3 into the joint quench Q.

In the regeneration mode, the heterogeneous particulate multimetal oxide catalyst is regenerated by passing an oxygen-comprising regeneration gas mixture over it and burning off the deposits deposited on the heterogeneous particulate multimetal oxide catalyst, with the n shell-and-tube reactors R1 to Rn preferably having a single heat transfer circuit and with at least one of the two or more shell-and-tube reactors always being operated in the production mode (production phase) so that the heat of reaction liberated minus the quantity of heat consumed for heating the feed gas stream to the reaction temperature in the production mode is sufficient to keep the temperature of the heat transfer medium in the intermediate spaces between the contact tubes of the two or more shell-and-tube reactors constant within a fluctuation range of not more than +/−10° C.

A shell-and-tube reactor Rm is run down by stopping the oxygen-comprising feed gas stream a2m to the shell-and-tube reactor Rm, throttling back the feed gas stream a1m comprising n-butenes to, for example, about ⅓ of the volume in the production phase and feeding in an inert gas stream a4m in such an amount that the resulting total gas flow through the reactor is, for example, still 64% of the volume of the total stream in the production phase. The volume of the inert gas a4m to be fed in is variable since the butane content of the feed stream a1m comprising n-butenes generally varies and the inert gas stream a4m is matched to this content. In the next step, the feed gas stream a1m comprising n-butenes is throttled back to, for example, ⅙ of the volume flow in the production phase, the recycle gas substream d2m is throttled back to, for example, 21% by volume of the total gas flow in the production phase and the inert gas stream a4m is increased to, for example, 39% by volume of the total gas flow in the production phase. In the next step, the feed gas stream a1m comprising n-butenes is shut off, whereupon the catalyst in the first shell-and-tube reactor Rm is regenerated. After, for example, from 10 to 30 minutes, the inert gas stream a4m is shut off and the first recycle gas stream d2m is increased to 90% by volume of the total gas flow in the production phase. The reoxidation of the catalyst subsequently takes place at an oxygen content of from 4 to 10% by volume, preferably over a period of from 12 to 19 hours. During this time, the first recycle gas stream d2m can be throttled back to 1%. The next production phase commences, starting from a recycle gas stream d2m of up to 90% by volume of the total gas flow in the production phase, by renewed introduction of the feed gas stream a1m comprising n-butenes, the oxygen-comprising feed gas stream a2m and throttling back of the first recycle gas stream d2m, in each case to the volume flows in the production phase.

In one variant, a substream of the recycle gas stream d2 is fed as compensation stream d3 to the quench Q in such a volume that the resulting total volume flow (b+d3) to quench Q, compressor K and absorption column A always remains approximately constant.

In a further variant, the product gas stream from a particular shell-and-tube reactor Rm at the end of the production mode thereof is discharged as stream qm and a substream of the recycle gas stream d2 having the same volume as the compensation stream d3 is fed to the Quench Q, as a result of which the total volume flow (b+d3) to quench Q, compressor K and absorption column A remains constant.

The process of the invention has numerous advantages. As a result of the use of a plurality of reactors with coupled heat transfer medium, the heat evolved in the oxydehydrogenation reactors can be exploited as heat source for the regeneration phase. Since at least one reactor is always in the production phase while another reactor is in the regeneration phase, it is ensured that the overall plant continuously produces desired product. The use of a plurality of reactors also means that recycle gas is continually obtained by removal of the incondensable or low-boiling gas constituents of the product gas from the oxydehydrogenation in a sufficient amount (1) to ensure a constant volume loading of the joint work-up by utilization of a compensation stream d3 and (2) for both the production phase and the regeneration phase of all shell-and-tube reactors to rest on substreams d2m of the joint recycle gas d2.

The Invention is based on the recognition that the regeneration of the catalyst can be divided into two phases each having different requirements in terms of the gas atmosphere, namely into an (a) burning-off phase and a (b) reoxidation phase. The burning-off of carbon-comprising compounds occurs within a few minutes, also with participation of lattice oxygen of the catalyst. This is followed by a phase of reoxidation. It has now surprisingly been found, contrary to DE 734026 and WO14086768, that if this phase does not continue for a sufficiently long time, the catalyst does not regain its full performance in the subsequent production cycle and the oxydehydrogenation reaction proceeds less selectively, so that the catalyst has an increased carbonization tendency compared to the previous production phase. The incorporation of molecular oxygen into the lattice structure of the catalyst and diffusion in the solid state is a slow process. In this phase, a relatively high oxygen content is advantageous since it promotes this slow process.

The process of the invention meets these requirements by the burning-off phase being carried out using a regeneration gas which is obtained by mixing part of the recycle gas substream d2m with an inert gas and has an oxygen content of from 2 to 3% by volume, preferably 2.3-2.7% by volume. Since the burning-off of carbon-comprising deposits occur successfully within a few minutes, the consumption of expensive inert gas can be reduced significantly and be restricted to the burning-off phase which takes only a few minutes in each case. After the burning-off of carbon-comprising deposits, which can be established by monitoring the COx content of the output steam, the reoxidation phase follows immediately. Since adhering carbon-comprising compounds have now been removed sufficiently, the associated restrictions placed on the oxygen content is order to protect the catalyst are unnecessary and it is even advantageous, according to the invention, to use the undiluted first recycle gas stream d21 having an oxygen content of preferably from 6.5 to 9% by volume as very advantageous regeneration gas for reoxidation of the catalyst.

Since the recycle gas d2 is obtained in compressed form, the existing pressure gradient can advantageously be exploited. Additional compressors as described, for instance, in WO 2015/007839 or WO 2015/007841 are not necessary.

A further advantage of the process of the invention is that flow occurs continually through the respective shell-and-tube reactor over all production and regeneration phases, so that outward transfer of heat via the gas stream is always ensured and very long residence times do not occur. High residence times in combination with reduced convection can lead to local hot spots and thus to a high thermal stress on the catalyst. During the burning-off phase, the total volume flow is reduced to, for example, 63% of the volume of flow during the production phase, as a result of which (1) the consumption of expensive inert gas is reduced further and (2) a still swift burning-off occurs due to the lower gas velocity. In particular, the gas velocity and the contents of oxygen and n-butenes are matched to one another during the running-down phase in such a way that neither does excessively rapid burning-off occur nor does carbonization of the catalyst occur due to an unselective reaction.

The above-described variant of the process of the invention in which the reactor output stream of a started-up reactor replaces the reactor output stream of the reactor to be run down in such a way that the total product gas stream b remains constant has the further advantage that not only does the volume load of the work-up remain approximately constant but the composition of the total product gas stream b also remains approximately constant.

As possible inert gases, preference is given to using nitrogen, but argon and CO₂ can also be used. Furthermore, steam can also be comprised in the oxygen-comprising regeneration gas mixture. Nitrogen is preferably used for setting the oxygen concentration, and the same applies to steam. Steam can also be present for removing the heat of reaction and as mild oxidant for the removal of carbon-comprising deposits. When steam is introduced into the reactor at the beginning of the regeneration, preference is given to using a proportional volume of 0-50% by volume, preferably 0-10% by volume and more preferably 0.1-7% by volume. The amount of nitrogen is selected so that the proportion by volume of molecular nitrogen in the regeneration gas mixture at the beginning of the burning-off phase is preferably 60-90% by volume and more preferably 60-65% by volume. Furthermore, the oxygen-comprising regeneration gas mixture can comprise hydrocarbons and reaction products of the oxidative dehydrogenation. The amount of the recycle gas substream d2m is selected so that the proportion of volume of molecular nitrogen in the regeneration gas mixture at the beginning of the burning-off phase is preferably 10-35% by volume and more preferably 30-35% by volume. In general, the total recycle gas stream d2 and correspondingly the recycle gas substream d2m comprise from 6.5 to 9% by volume of 02 and from 87 to 93% by volume of inert gases selected from among nitrogen, noble gases (in particular argon) and carbon oxides (in particular CO₂). In addition, the joint recycle gas stream d2 and correspondingly the recycle gas substream d2m can comprise from 0 to 2% by volume of carbon monoxide, from 0 to 1.5% by volume of oxygenates, e.g. acrolein, from 0 to 0.5% by volume of steam and from 0 to 0.1% by volume of hydrocarbons. The hydrocarbons can comprise saturated and unsaturated, branched and unbranched hydrocarbons such as methane, ethane, ethene, acetylene, propane, propene, propine, n-butane, isobutane, n-butene, isobutene, n-pentane and also dienes such as 1,3,5-butadiene and 1,2-butadiene. The oxygenates, hydrocarbons and carbon monoxide comprised have, in the presence of oxygen under the regeneration conditions in the presence of the catalyst, no reactivity or they are comprised in such small amounts that they interfere neither in the burning-off process nor in the reoxidation process.

According to the invention, a regeneration phase is carried out between each two production phases. The reactor under consideration can remain in the production mode (production stage) until the catalyst deactivation has reached a particular, prescribed value and, for example, the conversion at constant reaction temperature has dropped by 20%, preferably by 10% and particularly preferably by 5%. Furthermore, the reactor can remain in the production mode until the pressure drop over the reactor has risen by a particular, prescribed value, for example by 500 mbar, preferably by 100 mbar and particularly preferably by 25 mbar, or else a particular, prescribed duration of the production phase, for example 2000 hours, preferably 1000 hours, has elapsed.

The regeneration phase is preferably followed by a start-up phase comprising the steps vi) to viii):
vi) optionally additional feeding of a stream of steam a3m into the dehydrogenation zone;
vii) additional feeding-in of the feed gas stream a1m comprising butenes at a lower volume flow than in the production phase and increasing of this volume flow until at least 50% of the volume flow of the feed gas stream a1m in the production phase is reached, with the total gas flow through the reactor Rm corresponding to not more than 120% of the total gas flow during the production phase;
viii) additional feeding-in, when at least 50% of the volume flow of the feed gas stream a1m comprising butenes in the production phase is reached, of the oxygen-comprising stream a2m at a lower volume flow than in the production phase, and increasing of the volume flows of the gas streams a1m and a2m until the volume flows in the production phase have been reached, with the total gas flow through the dehydrogenation zone corresponding to not more than 120% of the total gas flow during the production phase.

The total gas stream comprising the streams a1m, a2m, d2m and optionally a3m through the dehydrogenation zone during steps (vi), (vii) and (viii) preferably is essentially constant and corresponds to from 90 to 110% by volume of the total gas flow through the dehydrogenation zone during the production phase.

Furthermore, the amount of steam in the dehydrogenation zone during steps vi), vii) and viii) is preferably from 0.5 to 10% by volume.

In general, the pressure in the reactor Rm during the start-up phase is from 1 to 5 bar.

The start-up procedure according to the invention has, compared to the mode of operation described in WO 2015/104397, the advantage that oxygen-rich conditions prevail even at the beginning of the start-up phase since the recycle gas is not diluted with an inert gas. As a result, carbonization of the catalyst is countered. The conditions during the start-up phase in respect of load, gas velocity, residence time and composition of the recycle gas stream tend to correspond to the conditions during the production phase. Since the gas velocity is substantially constant, a hot spot which is formed does not migrate within the reactor. The steady-state production state and the optimal performance of the catalyst in respect of space time yield and selectivity are reached overall more quickly.

In step vi), the recycle gas stream d2m is preferably set to from 90 to 110% of the total volume flow in the production phase. The total volume flow is the sum of the volume flows of a1m, a2m, d2m and optionally a3m. In a particularly preferred embodiment, the recycle gas stream d2m is set to 95-105% of the total volume flow in the production phase; particular preference is given to setting the recycle gas stream d2m to 100% of the total volume flow in the production phase. The recycle gas stream d2m set is reduced in the subsequent step vii) and viii) in such a way that the total gas flow through the dehydrogenation zone, i.e. the sum of the flows of a1m, a2m, d2m and optionally a3m, during the further start-up phase is at least 70% and not more than 120%, preferably at least 90% and not more than 115%, of the total gas flow during the production phase. The total gas flow during the start-up phase preferably remains substantially constant and varies by not more than +1-10% by volume, in particular +/−5% by volume, i.e. during the start-up phase is preferably from 90 to 110% by volume, in particular from 95 to 105% by volume, of the total gas flow during the production phase.

The gas stream d2′ has a composition corresponding to the recycle gas stream d2 in the production phase when its oxygen content deviates by not more than +1-2% by volume from the oxygen content of the recycle gas stream d2 in the steady-state production phase.

In step vi), a stream of steam a3m can additionally be fed into the reactor Rm. In general, the amount of steam during steps vi) to viii) is from 0.5 to 10% by volume, preferably from 1 to 7% by volume. This can also be atmospheric moisture.

In step vii), the feed gas stream a1m comprising butenes is additionally fed in until at least 50% of the volume flow in the production phase of the reactor has been reached. The volume flow is generally increased stepwise, for example, commencing at 10% of the volume flow in the production state, in steps of 10% until at least 50% of the volume flow in the production state is reached. The volume flow can also be increased in the form of a ramp. The recycle gas stream d2 is optionally reduced correspondingly to such an extent that the total gas flow through the reactor Rm corresponds to not more than 120% of the total gas flow during the production phase.

The content of C₄-hydrocarbons (butenes and butanes) in the total gas stream through the reactor Rm at the end of step (vii) is generally from 7 to 9% by volume.

The volume flow of the feed gas stream a1m comprising butenes can also be increased in step vii) until at least 60% of the volume flow in the production phase has been reached, but at most until a maximum of 75% of the volume flow in the production phase has been reached.

In step viii), when at least 50% and not more than 75% of the volume flow of the feed gas stream a1m comprising butenes in the production phase have been reached, an oxygen-comprising stream a2m having a smaller volume flow than in the production phase is fed in addition to the feed gas stream a1m comprising butenes into the dehydrogenation zone and the volume flows of the feed gas streams a1m and a2m are increased until the volume flows in the production phase have been reached. In general, the volume flow of the oxygen-comprising gas stream a2m is, in a first step, increased in one or more stages until a ratio of oxygen to hydrocarbons which corresponds to the ratio of oxygen to hydrocarbons in the production phase has been reached, and both volume flows of a1m and a2m are subsequently increased in stages until in each case 100% of the volume flow of the gas streams a1m and a2m in the production phase has been reached, with the ratio of oxygen to hydrocarbons remaining substantially constant and corresponding to the ratio of oxygen to hydrocarbons in the production phase. The volume flow is generally increased stepwise, for example commencing at 50% of the volume flow of the gas stream a1m in the production state, in steps of, for example, 10%, with the steps for the stages for increasing the volume flow of the gas stream a2m being selected so that the ratio of oxygen to hydrocarbons during the start-up phase remains substantially constant until 100% of the volume flows in the production state has been reached.

At the end of step viii), the content of C₄-hydrocarbons (butenes and butanes) in the total gas stream through the reactor Rm is generally from 7 to 9% by volume, and the oxygen content is generally from 12 to 13% by volume.

In general, the pressure in the dehydrogenation zone during the start-up phase is from 1 to 5 bar absolute, preferably from 1.05 to 2.5 bar absolute.

In general, the pressure in the absorption zone during the start-up phase is from 2 to 20 bar, preferably from 5 to 15 bar.

In general, the temperature of the heat transfer medium during the start-up phase is in the range from 220 to 490° C. and preferably from 300 to 450° C. and particularly preferably from 330 to 420° C.

In general, the duration of the start-up phase is from 15 to 2000 minutes, preferably from 15 to 500 minutes and particularly preferably from 20 to 120 minutes. The production phase then commences.

In general, the step C) comprises the steps Ca) and Cb):
Ca) cooling of the product gas stream b in at least one cooling stage, with cooling in at least one cooling stage being effected by contacting with a coolant, and condensation of at least part of the high-boiling secondary components;
Cb) compression of the remaining product gas stream b in at least one compression stage, giving at least one aqueous condensate stream c1 and a gas stream c2 comprising butadiene, n-butenes, water vapor, oxygen, low-boiling hydrocarbons, possibly carbon oxides and possibly inert gases.

In general, the step D) comprises the steps Da) and Db):
Da) separation of incondensable and low-boiling gas constituents comprising oxygen, low-boiling hydrocarbons, possibly carbon oxides and possibly inert gases as gas stream d from the gas stream c2 by absorption of the C₄-hydrocarbons comprising butadiene and n-butenes in an absorption medium, giving an absorption medium stream loaded with C₄-hydrocarbons and gas stream d, and
Db) a subsequent desorption of the C₄-hydrocarbons from the loaded absorption medium stream, giving a C₄ product gas stream d1.

The steps E) and F) are preferably also carried out subsequently:
E) separation of the C₄ product stream d1 by extractive distillation using a solvent which is selective for butadiene into a stream e1 comprising butadiene and the selective solvent and a stream e2 comprising n-butenes;
F) distillation of the stream f2 comprising butadiene and the selective solvent into a stream g1 consisting essentially of the selective solvent and a stream g2 comprising butadiene.

In general, the gas stream d obtained in step Da) is recirculated to an extent of at least 10%, preferably at least 30%, as recycle gas stream d2 to step B).

In general, aqueous coolants or organic solvents or mixtures thereof are used in the cooling stage Ca).

Preference is given to using an organic solvent in the cooling stage Ca). Such a solvent generally has a very much higher solvent capability for the high-boiling secondary products which can lead to deposits and blockages in the plant parts downstream of the ODH reactor than for water or alkaline aqueous solutions. Preferred organic solvents used as coolants are aromatic hydrocarbons, for example toluene, o-xylene, n-xylene, p-xylene, diethylbenzenes, triethylbenzenes, diisopropylbenzenes, triisopropylbenzenes and mesitylene or mixtures thereof. Particular preference is given to mesitylene.

The following embodiments are preferred or particularly preferred variants of the process of the invention:

The step Ca) is carried out in a plurality of stages Ca1) to Can), preferably in two stages Ca1) and Ca2). Here, particular preference is given to at least part of the solvent which has passed through the second stage Ca2) being fed as coolant to the first stage Ca1).

The step Cb) generally comprises at least one compression stage Cba) and at least one cooling stage Cbb). The gas which has been compressed in the compression stage Cba) is preferably brought into contact with a coolant in the at least one cooling stage Cbb). The coolant in the cooling stage Cbb) particularly preferably comprises the same organic solvent which is used as coolant in the step Ca). In a particularly preferred variant, at least part of this coolant is, after passing through the at least one cooling stage Cbb), fed as coolant to the step Ca).

The step Cb) preferably comprises a plurality of compression stages Cba1) to Cban) and cooling stages Cbb1) to Cbbn), for example four compression stages Cba1) to Cba4) and four cooling stages Cbb1) to Cbb4).

Step D) preferably comprises the steps Da1), Da2) and Db):
Da1) absorption of the C₄-hydrocarbons comprising butadiene and n-butenes in a high-boiling absorption medium, giving an absorption medium stream loaded with C₄-hydrocarbons and the gas stream d,
Da2) removal of oxygen from the absorption medium stream loaded with C₄-hydrocarbons from step Da) by stripping with an incondensable gas stream, and
Db) desorption of the C₄-hydrocarbons from the loaded absorption medium stream, giving a C₄ product gas stream d1 which consist essentially of C₄-hydrocarbons and comprises less than 100 ppm of oxygen.

The high-boiling absorption medium used in step Da) is preferably an aromatic hydrocarbon solvent, particularly preferably the aromatic hydrocarbon solvent used in step Ca), in particular mesitylene. It is also possible to use, for example diethylbenzenes, triethylbenzenes, diisopropylbenzenes and triisopropylbenzenes or mixtures comprising these substances.

Embodiments of the process of the invention are depicted in FIG. 1 and are described in detail below.

As feed gas stream, it is possible to use pure n-butenes (1-butene and/or cis-/trans-2-butene) or else gas mixtures comprising butenes. It is also possible to use a fraction which comprises n-butenes (1-butene and cis-/trans-2-butene) as main constituent and has been obtained from the C₄ fraction from naphtha cracking by removal of butadiene and isobutene. Furthermore, it is also possible to use gas mixtures which comprise pure 1-butene, cis-2-butene, trans-2-butene or mixtures thereof and have been obtained by dimerization of ethylene as feed gas. Furthermore, it is possible to use gas mixtures which comprise n-butenes and have been obtained by fluid catalytic cracking (FCC) as feed gas.

In one embodiment of the process of the invention, the feed gas comprising n-butenes is obtained by nonoxidative dehydrogenation of n-butane. As a result of the coupling of a nonoxidative catalytic dehydrogenation with the oxidative dehydrogenation of the n-butenes formed, it is possible to obtain a high yield of butadiene, based on n-butane used. The nonoxidative catalytic dehydrogenation of n-butane gives a gas mixture comprising butadiene, 1-butene, 2-buten and unreacted n-butane together with secondary constituents. Typical secondary constituents are hydrogen, water vapor, nitrogen, CO and CO₂, methane, ethane, ethene, propane and propene. The composition of the gas mixture leaving the first dehydrogenation zone can very greatly de-pending on the mode of operation of the dehydrogenation. Thus, when the dehydrogenation is carried out with introduction of oxygen and additional hydrogen, the product gas mixture has a comparatively high content of water vapor and carbon oxides. In modes of operation without introduction of oxygen, the product gas mixture from the nonoxidative dehydrogenation has a comparatively high content of hydrogen.

In step B) the feed gas stream comprising n-butenes and an oxygen-comprising gas are fed into at least one dehydrogenation zone (one or more ODH reactors R operated in parallel) and the butenes comprised in the gas mixture are oxidatively dehydrogenated to butadiene in the presence of an oxydehydrogenation catalyst.

The gas comprising molecular oxygen generally comprises more than 10% by volume, preferably more than 15% by volume and even more preferably more than 20% by volume, of molecular oxygen. It is preferably air. The upper limit to the content of molecular oxygen is generally 50% by volume or less, preferably 30% by volume or less and even more preferably 25% by volume or less. In addition, any Inert gases can be comprised in the gas comprising molecular oxygen. As possible inert gases, mention may be made of nitrogen, argon, neon, helium, CO, CO₂ and water. The amount of inert gases is in the case of nitrogen generally 90% by volume or less, preferably 85% by volume or less and even more preferably 80% by volume or less. In the case of constituents other than nitrogen, it is generally 10% by volume or less, preferably 1% by volume or less.

To carry out the oxidative dehydrogenation at complete conversion of n-butenes, preference is given to a gas mixture having a molar oxygen:n-butenes ratio of at least 0.5. Preference is given to working at an oxygen:n-butenes ratio of from 1.25 to 1.6. To set this value, the feed gas stream can be mixed with oxygen or at least one oxygen-comprising gas, for example air, and optionally additional inert gas or steam. The oxygen-comprising gas mixture obtained is then fed to the oxydehydrogenation.

Furthermore, inert gases such as nitrogen and also water (as water vapor) can also be comprised together in the reaction gas mixture. Nitrogen can serve for setting the oxygen concentration and for preventing the formation of an explosive gas mixture; the same applies to steam. Steam also serves for control of the carbonization of the catalyst and for removal of the heat of reaction.

Catalysts suitable for the oxydehydrogenation are generally based on an Mo—Bi—O-comprising multimetal oxide system which generally additionally comprises iron. In general, the catalyst comprises further additional components such as potassium, cesium, magnesium, zirconium, chromium, nickel, cobalt, cadmium, tin, lead, germanium, lanthanum, manganese, tungsten, phosphorus, cerium, aluminum or silicon. Iron-comprising ferrites have also been proposed as catalysts.

In a preferred embodiment, the multimetal oxide comprises cobalt and/or nickel. In a further professed embodiment, the multimetal oxide comprises chromium. In a further preferred embodiment, the multimetal oxide comprise manganese.

Examples for Mo—Bi—Fe—O-comprising multimetal oxides are Mo—Bi—Fe—Cr—O— or Mo—Bi—Fe—Zr—O-comprising multimetal oxides. Preferred catalysts are, for example, described in U.S. Pat. No. 4,547,615 (Mo₁₂BiFe_{0.1}Ni₈ZrCr₃K_{0.2}Oₓ and Mo₁₂BiFe_{0.1}Ni₈AlCr₃K_{0.2}Oₓ), U.S. Pat. No. 4,424,141 (Mo₁₂BiFe₃Co_{4.5}Ni_{2.5}P_{0.5}K_{0.1}Oₓ+SiO₂), DE-A 25 30 959 (Mol2BiFe₃Co_{4.5}Ni_{2.6}Cr_{0.5}K_{0.1}Oₓ. Mo_{13.75}BiFe₃Co_{4.5}Ni_{2.5}Ge_{0.5}K_{0.8}Oₓ. Mo₁₂BiFe₃Co_{4.5}Ni_{2.5}Mn_{0.5}K_{0.1}Oₓ and Mo₁₂BiFe₃Co_{4.5}Ni_{2.5}La_{0.5}K_{0.1}Oₓ), U.S. Pat. No. 3,911,039 (Mo₁₂BiFe₃Co_{4.5}Ni_{2.5}Sn_{0.5}K_{0.1}Oₓ), DE-A 25 30 959 and DE-A 24 47 825 (Mo₁₂BiFe₃Co_{4.5}Ni_{2.5}W_{0.5}K_{0.1}Oₓ).

Suitable multimetal oxides and the preparation thereof are also described in U.S. Pat. No. 4,423,281 (Mo₁₂BiNi₈Pb_{0.5}Cr₃K_{0.2}Oₓ and Mo₁₂BiNi₇Al₃Cr_{0.5}K_{0.5}Oₓ), U.S. Pat. No. 4,336,409 (Mo₁₂BiNi₆Cd₂Cr₃P_{0.5}Oₓ), DE-A 26 00 128 (Mo₁₂BiN_{0.5}Cr₃P_{0.5}Mg_{7.5}K_{0.1}Oₓ+SiO₂) and DE-A 24 40 329 (Mo₁₂BiCo_{4.5}Ni_{2.5}Cr₃P_{0.5}K_{0.1}Oₓ).

Particularly preferred catalytically active multimetal oxides comprising molybdenum and at least one further metal have the general formula (Ia):

Mo₁₂BiₐFe_{b}Co_{c}Ni_{d}CrₑX¹_{f}X²_{g}O_{y} (Ia),

where
X¹=Si, Mn and/or Al,
X²=Li, Na, K, Cs and/or Rb,
0.2≤a≤1,
0.5≤b≤10,
0≤c≤10,
0≤d≤10,
2≤c+d≤10
0≤e≤2,
0≤f≤10
0≤g≤0.5
y=a number which is determined by the valance and abundance of the elements other than oxygen in (Ia) in order to achieve charge neutrality.

Preference is given to catalysts whose catalytically active oxide composition comprises only Co from among the two metals Co and Ni (d=0). X¹ is preferably Si and/or Mn and X² is preferably K, Na and/or Cs, with particular preference being given to X²═K. A largely Cr(VI)-free catalyst is particularly preferred.

The reaction temperature in the oxydehydrogenation is generally controlled by means of a heat transfer medium which is present around the reaction tubes. Possible liquid heat transfer media of this type are, for example, melts of salts or salts mixtures such as potassium nitrate, potassium nitrite, sodium nitrite and/or sodium nitrate and also melts of metals such as sodium, mercury and alloys of various metals. However, ionic liquids or heat transfer oils can also be used. The temperature of the heat transfer medium is in the range from 220 to 490° C. and preferably from 300 to 450° C. and particularly preferably from 330 to 420° C.

Owing to the exothermic nature of the reactions which proceed, the temperature in particular sections of the reactor interior during the reaction can be higher than that of the heat transfer medium and a hot spot is formed. The position and magnitude of the hot spot is determined by the reaction conditions, but can also be regulated by the dilution ratio of the catalyst zone or the passage of mixed gas. The difference between hot spot temperature and the temperature of the heat transfer medium is generally in the range from 1 to 150° C., preferably from 10 to 100° C. and particularly preferably from 20 to 80° C. The temperature at the end of the catalyst bed is generally from 0 to 100° C. above, preferably from 0.1 to 50° C. above, particularly preferably from 1 to 25° C. above, the temperature of the heat transfer medium.

The oxydehydrogenation can be carried out in all fixed-bed reactors which are known from the prior art, for example in a tray oven, in a fixed-bed tube reactor or shell-and-tube reactor or in a plate heat exchanger reactor. A shell-and-tube reactor is preferred.

The oxidative dehydrogenation is preferably carried out in fixed-bed tube reactors or fixed-bed shell-and-tube reactors. The reaction tubes are (like the other elements of the shell-and-tube reactor) generally made of steel. The wall thickness of the reaction tubes is typically from 1 to 3 mm. Their internal diameter is generally (uniformly) from 10 to 50 mm or from 15 to 40 mm, frequently from 20 to 30 mm. The number of reaction tubes accommodated in the shell-and-tube reactor is generally at least 1000, or 3000, or 5000, preferably at least 10000. The number of reaction tubes accommodated in the shell-and-tube reactor is frequently from 15000 to 30000 or up to 40000 or up to 50000. The length of the reaction tubes is normally a few meters, with a reaction tube length in the range from 1 to 8 m, frequently from 2 to 7 m, often form 2.5 to 6 m, being typical.

Furthermore, the catalyst bed which is installed in the ODH reactor(s) R can consist of a single zone or of two or more zones. These zones can consist of pure catalyst or be diluted with a material which does not react with the feed gas or components of the product gas from the reaction. Furthermore, the catalyst zones can consist of all-active material and/or supported coated catalysts.

The product gas stream leaving the oxidative dehydrogenation comprises not only butadiene but generally also unreacted 1-butene and 2-butene, oxygen and water vapor. As secondary components, it generally further comprises carbon monoxide, carbon dioxide, inert gases (mainly nitrogen), low-boiling hydrocarbons such as methane, ethane, ethene, propane and propene, butane and isobutane, possibly hydrogen and possibly oxygen-comprising hydrocarbons, known as oxygenates. Oxygenates can be, for example, formaldehyde, furan, acetic acid, maleic anhydride, formic acid, methacrolein, methacrylic acid, crotonaldehyde, crotonic acid, propionic acid, acrylic acid, methyl vinyl ketone, styrene, benzaldehyde, benzoic acid, phthalic anhydride, fluorenone, anthraquinone and butyraldehyde.

The product gas stream at the reactor outlet has a temperature close to the temperature at the end of the catalyst bed. The product gas stream is then brought to a temperature of from 150 to 400° C., preferably from 160 to 300° C., particularly preferably from 170 to 250° C. It is possible to insulate the conduit through which the product gas flows in order to keep the temperature in the desired range or to use a heat exchanger. This heat exchanger system can be of any type as long as the temperature of the product gas can be kept at the desired level by means of the system. As examples of a heat exchanger, mention may be made of helical heat exchangers, plate heat exchangers, double-tube heat exchangers, multitube heat exchangers, tank-helical heat exchangers, tank-jacket heat exchangers, liquid-liquid-contact heat exchangers, air heat exchangers, direct contact heat exchangers and finned tube heat exchangers. Since part of the high-boiling by-products comprised in the product gas can precipitate while the temperature of the product gas is brought to the desired temperature, the heat exchanger system should preferably have two or more heat exchangers. If two or more heat exchangers provided are arranged in parallel and divided cooling of the obtained product gas in the heat exchangers is thus made possible, the amount of high-boiling by-products which deposit in the heat exchangers decreases and the period of operation thereof can thus be extended. As an alternative to the above-mentioned method, the two or more heat exchangers provided can be arranged in parallel. The product gas is fed to one or more, but not all, heat exchangers which are relieved after a particular period of operation by other heat exchangers. In this method, cooling can be continued, part of the heat of reaction can be recovered and, parallel thereto, the high-boiling by-products deposited in one of the heat exchangers can be removed. As a coolant of the type mentioned above, it is possible to use a solvent as long as it is able to dissolve the high-boiling by-products. Examples are aromatic hydrocarbon solvents such as toluene and xylenes, diethylbenzenes, triethylbenzenes, diisopropylbenzenes, triisopropylbenzenes. Particular preference is given to mesitylene. It is also possible to use aqueous solvents. These can be made either acidic or alkaline, for example an aqueous solution of sodium hydroxide.

A major part of the high-boiling secondary components and of the water is subsequently separated off from the product gas stream by cooling and compression. Cooling is effected by contacting with a coolant. This stage will hereinafter also be referred to as quench Q. This quench can consist of only one stage or of a plurality of stages. The product gas stream is thus brought into contact directly with a preferably organic cooling medium and cooled thereby. Suitable cooling media are aqueous coolants or organic solvents, preferably aromatic hydrocarbons, particularly preferably toluene, o-xylene, m-xylene, p-xylene or mesitylene, or mixtures thereof. All possible isomers of diethylbenzene, triethylbenzene, diisopropylbenzene and triisopropylbenzene and mixtures thereof can also be used.

Preference is given to a two-stage quench, i.e. the step Ca) comprises two cooling stages Ca1) and Ca2) in which the product gas stream b is brought into contact with the organic solvent.

Thus, in a preferred embodiment of the invention, the cooling step Ca) is carried out in two stages, with the solvent loaded with secondary components from the second stage Ca2) being fed into the first stage Ca1). The solvent taken off from the second stage Ca2) comprises a smaller amount of secondary components than the solvent taken off from the first stage Ca1).

A gas stream comprising n-butane, 1-butene, 2-butenes, butadiene, possibly oxygen, hydrogen, water vapor, small amounts of methane, ethane, ethene, propane and propene, isobutene, carbon oxides, inert gases and amounts of the solvent used in the quench is obtained. Furthermore, traces of high-boiling components which have not been quantitively be separated off in the quench can remain in this gas stream.

The product gas stream from the solvent quench is compressed in at least one compression stage K and subsequently cooled further in the cooling apparatus, forming at least one condensate stream. A gas stream comprising butadiene, 1-butene, 2-butenes, oxygen, water vapor, possibly low-boiling hydrocarbons such as methane, ethane, ethene, propane and propene, butane and isobutane, possibly carbon oxides and possibly inert gases remains. Furthermore, this product gas stream can comprise traces of high-boiling components.

The compression and cooling of the gas stream can be carried out in one or more stages (n-stage). In general, compression is carried out overall from a pressure in the range from 1.0 to 4.0 bar (absolute) to a pressure in the range from 3.5 to 20 bar (absolute). Each compression stage is followed by a cooling stage in which the gas stream is cooled to a temperature in the range from 15 to 60° C. The condensate stream can thus also comprise a plurality of streams in the case of multiple compression. The condensate stream consists largely of water and possibly the organic solvent used in the quench. The two streams (aqueous and organic phases) can also comprise small amounts of secondary components such as low boilers, C₄-hydrocarbons, oxygenates and carbon oxides.

The gas stream comprising butadiene, n-butenes, oxygen, low-boiling hydrocarbons (methane, ethane, ethene, propane, propene, n-butane, isobutane), possibly water vapor, possibly carbon oxides and possibly inert gases and possibly traces of secondary components is passed as output stream to further work-up.

In a step D), incondensable and low-boiling gas constituents comprising oxygen, low-boiling hydrocarbons (methane, ethane, ethene, propane, propene), carbon oxides and inert gases are separated off as gas stream from the process gas stream by absorption of the C₄-hydrocarbons in a high-boiling absorption medium in an absorption column A and subsequent desorption of the C₄-hydrocarbons. The step D) preferably comprises the steps Da1), Da2) and Db):
Da1) absorption of the C₄-hydrocarbons comprising butadiene and n-butenes in a high-boiling absorption medium, giving an absorption medium stream loaded with C₄-hydrocarbons and the gas stream,
Da2) removal of oxygen from the absorption medium stream loaded with C₄-hydrocarbons from step Da) by stripping with an incondensable gas stream, giving an absorption medium stream loaded with C₄-hydrocarbons, and
Db) desorption of C₄-hydrocarbons from the loaded absorption medium stream, giving a C₄-product gas stream which consists essentially of C₄-hydrocarbons.

For this purpose, the gas stream is brought into contact with an inert absorption medium in the absorption step Da1) and the C₄-hydrocarbons are absorbed in the inert absorption medium, giving an absorption medium loaded with C₄-hydrocarbons and an offgas comprising the remaining gas constituents. In a desorption stage, the C₄-hydrocarbons are liberated again from the high-boiling absorption medium.

The absorption step can be carried out in any suitable absorption column known to those skilled in the art. The absorption can be effected by simply passing the product gas stream through the absorption medium. However, it can also be carried out in columns or in rotary absorbers. The absorption can here be carried out in cocurrent, countercurrent or cross-current. The absorption is preferably carried out in countercurrent. Suitable absorption columns are, for example, tray columns having bubble cap trays, centrifugal trays and/or sieve trays, columns having structured packing, e.g. sheet metal packing having a specific surface area of from 100 to 1000 m²/m³, e.g. Mellapak® 250 Y, and columns comprising random packing elements. However, trickle and spray towers, graphite block absorbers, surface absorbers such as thick-film and thin-film absorbers and also rotary columns, plate scrubbers, crossed-spray scrubbers and rotational scrubbers are also possible.

In one embodiment an absorption column is supplied in the lower region with the gas stream comprising butadiene, n-butenes and the low-boiling and incondensable gas constituents. The high-boiling absorption medium is fed to the upper region of the absorption column.

Inert absorption media used in the absorption stage are generally high-boiling nonpolar solvents in which the C₄-hydrocarbon mixture to be separated off has a significantly higher solubility than the remaining gas constituents to be separated off. Suitable absorption media are comparatively nonpolar organic solvents, for example aliphatic C₈-C₁₈-alkanes, or aromatic hydrocarbons such as the middle oil fractions from paraffin distillation, toluene or ethers having bulky groups or mixtures of these solvents, with a polar solvent such as 1,2-dimethyl phthalate being able to be added to these. Further suitable absorption media are esters of benzoic acid and phthalic acid with straight-chain C₁-C₈-alkanols, and also heat transfer oils such as biphenyl and diphenyl ether, chloro-derivatives thereof and also triarylalkenes. One suitable absorption medium is a mixture of biphenyl and diphenyl ether, preferably in the azeotropic composition, for example the commercially available Diphyl®. This solvent mixture frequently comprises dimethyl phthalate in an amount of from 0.1 to 25% by weight.

In a preferred embodiment of the absorption stage Da1), the same solvent as in the cooling stage Ca) is used.

Preferred absorption media are solvents which have a solvent capability for organic peroxides of at least 1000 ppm (mg of active oxygen/kg of solvent). Preference is given to aromatic hydrocarbons, particularly preferably toluene, o-xylene, p-xylene and mesitylene, or mixtures thereof. All possible isomers of diethylbenzene, triethylbenzene, diisopropylbenzene and triisopropylbenzene and mixtures thereof can also be used.

A gas stream d comprising essentially oxygen, low-boiling hydrocarbons (methane, ethane, ethene, propane, propene), the hydrocarbon solvent, possibly C₄-hydrocarbons (butane, butene, butadiene), possibly inert gases, possibly carbon oxides and possibly water vapor is taken off at the top of the absorption column. This stream is at least partly fed as recycled gas stream d2 to the ODH reactor. In this way, it is possible, for example, to set the inflow stream of the ODH reactor to the desired C₄-hydrocarbon content. In general, optionally after separating off a purge gas stream, at least 10% by volume, preferably at least 30% by volume, of the gas stream d is recirculated as recycle gas stream d2 to the oxidative dehydrogenate zone.

In general, the recirculated stream amounts to from 10 to 70% by volume, preferably from 30 to 60% by volume, of the sum of all streams fed into the oxidative dehydrogenation B).

The purge gas stream can be subjected to thermal or catalytic after-combustion. In particular, it can be utilized thermally in a power station.

At the bottom of the absorption column, residues of oxygen dissolved in the absorption medium are discharged in a further column by flushing with a gas. The remaining proportion of oxygen should be so small that the stream which leaves the desorption column and comprises butane, butene and butadiene comprises a maximum of 100 ppm of oxygen.

The stripping-out of the oxygen in step Da2) can be carried out in any suitable column known to those skilled in the art. Stripping can be effected by simply passing incondensable gases, preferably gases which are not absorbable or only slightly absorbable in the absorption medium stream, e.g. methane, through the loaded absorption solution. C₄-hydrocarbons which are stripped out are scrubbed back into the absorption solution in the upper part of the column by the gas stream being conveyed back into this absorption column. This can be effected both by piping of the stripper column and also by direct installation of the stripper column underneath the absorber column. Since the pressure in the stripping column section and the absorption column section is the same, this direct coupling can be achieved. Suitable stripping columns are, for example, tray columns having bubble cap trays, centrifugal trays and/or sieve trays, columns having structured packing, e.g. sheet metal packing having a specific surface area of from 100 to 1000 m²/m³, e.g. Mellapak® 250 Y, and columns comprising random packing elements.

However, trickle and spray towers and also rotary columns, plate scrubbers, crossed-spray scrubbers and rotational scrubbers are also possible. Suitable gases are, for example nitrogen or methane.

In one embodiment of the process, stripping is carried out in step Da2) using a methane-comprising gas stream. In particular, this gas stream (stripping gas) comprises >90% by volume of methane.

The absorption medium stream loaded with C₄-hydrocarbons can be heated in a heat exchanger and subsequently introduced into a desorption column. In one process variant, the desorption step Db) is carried out by depressurization and stripping of the loaded absorption medium by means of a stream of steam.

The absorption medium which has been regenerated in the desorption stage can be cooled in a heat exchanger. The cooled stream comprises the absorption medium together with water which is separated off in the phase separator.

The C₄ product gas stream consisting essentially of n-butane, n-butenes and butadiene generally comprises from 20 to 80% by volume of butadiene, from 0 to 80% by volume or n-butane, from 0 to 10% by volume of 1-butene, from 0 to 50% by volume of 2-butenes and from 0 to 10% by volume of methane, with the total amount adding up to 100% by volume. Furthermore, small amounts of isobutane can be comprised.

Part of the condensed overhead output comprising mainly C₄-hydrocarbons from the desorption column can be recirculated into the top of the column in order to increase the separation performance of the column.

The liquid or gaseous C₄ product streams leaving the condenser can subsequently be separated by extractive distillation in the step E) using a butadiene-selective solvent into a stream comprising butadiene and the selective solvent and a stream comprising butanes and n-butenes.

### EXAMPLE

The tube reactor (R) consists of stainless steel 1.4571, has an internal diameter of 29.7 mm and a length of 5 m and is filled with a mixed oxide catalyst (2500 ml). A thermocouple sheath (external diameter 6 mm) having a thermocouple located therein is installed in the center of the tube in order to measure the temperature profile in the bed. A salt melt flows around the tube in order to keep the outer wall temperature constant. A stream of butenes and butanes (a1), steam (a3), air (a2) and oxygen-comprising recycle gas (d2) is fed to the reactor. Furthermore, nitrogen (a4) can be fed into the reactor.

The product gas (b) is cooled to 45° C. in a quenching apparatus (Q), with the high-boiling by-products being separated off. The stream is compressed in a compressor stage (K) to 10 bar and cooled again to 45° C. In the cooler, a condensate stream (c1) is discharged. The gas stream (c2) is fed to an absorption column (A). The absorption column is operated using mesitylene. From the absorption column, a liquid stream rich in organic products and a gaseous stream (d) at the top of the absorption column are obtained. The total work-up is designed so that water and the organic components are separated off completely. Part of the stream (d) is fed back as recycle gas (d2) to the reactor.

### Example 1

FIG. 1 schematically shows the experimental setup, with only one reactor R being considered. The oxygen concentration in the recycle gas d2 in the steady-state production state is 7.5% by volume. In the steady-state production state, the reactor Is supplied with:
a stream a consisting of 85% by volume of butenes and 15% by volume of butanes, with a flow of 516 standard I/h,
a stream a2, in this case air, having a flow of 2431 standard I/h,
a stream a3, steam, having a flow of 275 standard I/h and
a stream of recycle gas d2 having a flow of 2278 standard I/h.

This gives a total gas flow in the steady-state production state of 5500 standard I/h.

The running down phase is commenced by, over a period of 10 minutes, the stream a2 being shut off and the stream a1 being throttled back to 172 standard I/h and the stream a3 being throttled back to 175 standard I/h and inert gas being fed in as stream a4 having a flow of 875 standard I/h.

Subsequently, over a period of 10 minutes, the inert gas stream a4 is increased to 2175 standard I/h, the recycle gas stream d2 is throttled back to 1150 standard I/h and the stream a1 is throttled back to 86 standard I/h.

The stream a1 is then shut off. At the reactor inlet, a final value of 2.5% by volume of oxygen is obtained, with the oxygen originating from the recycle gas stream d2.

The inert gas stream a4 is then shut off and the recycle gas stream d2 is increased to 3325 standard I/h.

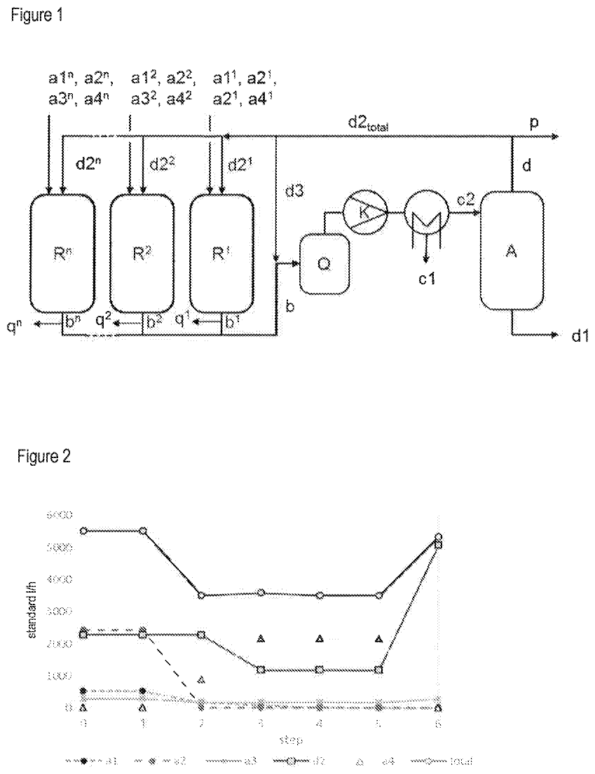

FIG. 2 shows the introduction scheme for the reactor tube with the phases: production phase (steps 0 and 1), running down phase (steps 1 to 4), burning-off phase (steps 4 and 5) and reoxidation phase (steps 5 and 6).

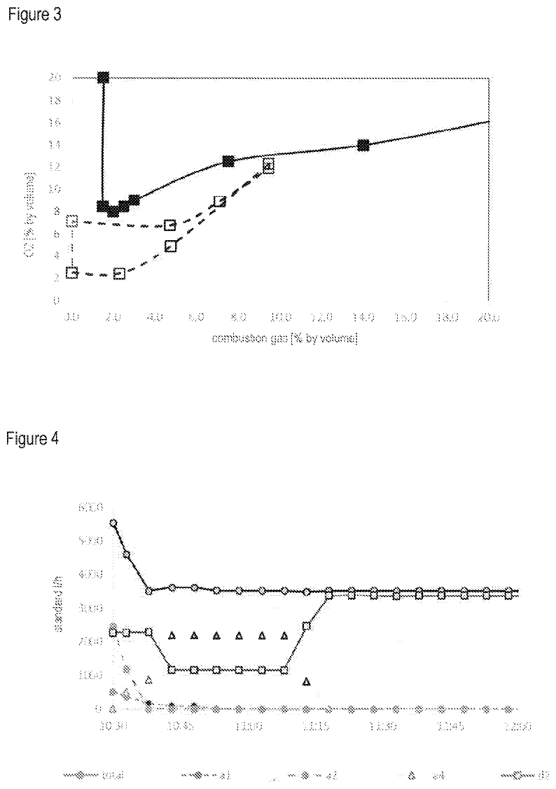

FIG. 3 shows the explosion graph for the introduction scheme.

FIG. 4 shows the volume flows in standard I/h of C₄-hydrocarbons a1, oxygen-comprising gas a2, recycle gas stream d2 and also the inert gas stream (nitrogen) a4 during the running-down and regeneration procedure according to the invention. The O₂ concentration in the feed is initially 12.3% by volume in order to simulate the O₂ concentration in the recycle gas stream d2.

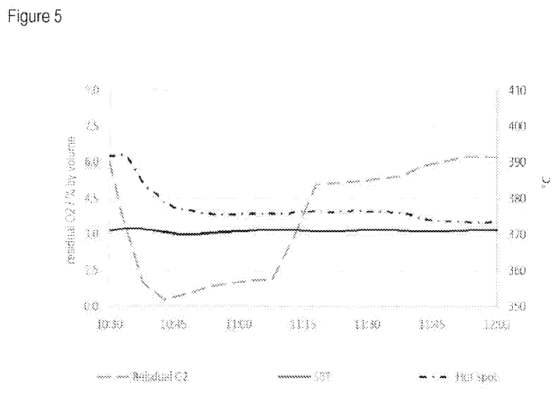

FIG. 5 shows the residual oxygen content obtained at the outlet from the quenching stage Q in % by volume, the salt bath temperature SBT and the hot spot temperature in ° C. during the running-down and regeneration procedure according to the invention, corresponding to the streams shown in FIG. 4. The O₂ concentration in the feed is initially 12.3% by volume in order to simulate the O₂ concentration in the recycle gas stream d2. The hot spot gradually decreases during running-down, and neither increases greatly nor decreases greatly in a short time. Immediately before commencement of running down, the oxygen concentration downstream of the reactor is 6.0% by volume and that in the recycle gas is 7.5% by volume. During running down, the value downstream of the reactor decreases. Due to the burning-off of carbonaceous material or carbon-comprising compounds, the value is below the oxygen concentration of 2.5% by volume temporarily set at the reactor inlet. After the nitrogen stream has been shut off and the recycle gas stream has been increased again, the oxygen concentration increases downstream of the reactor, due to dilution with steam, to 7.1% by volume or to 7.5% by volume between quench and absorption column and in the recycle gas. After the introduction of air has been shut off, the oxygen concentration cannot exceed the value in the recycle gas both upstream and downstream of the reactor and between quenching column and absorption column. Safe running down which is gentle on the catalyst is thus ensured.

1. A process for preparing butadiene from n-butenes in n reactors R1 to Rn operated in parallel, with each of the plurality of reactors operating in parallel going through a production phase and a regeneration phase, wherein the process in the production phase of a reactor Rm in the n reactors comprises the steps:
A) provision of a feed gas stream a1m comprising n-butenes;
B) feeding of the feed gas stream a1m comprising n-butenes, an oxygen-comprising gas stream a2m and a substream d2m of an oxygen-comprising total recycle gas stream d2 into the oxidative dehydrogenation zone of the reactor and oxidative dehydrogenation of n-butenes to butadiene, giving a product gas substream bm comprising butadiene, unreacted n-butenes, water vapor, oxygen, low-boiling hydrocarbons, high-boiling secondary components, possibly carbon oxides and possibly inert gases;
C) combination of the product gas substream bm with further product gas substreams to form a total product gas stream b and cooling and compression of the total product gas stream b and condensation of at least part of the high-boiling secondary components, giving at least one aqueous condensate stream c1 and a gas stream c2 comprising butadiene, n-butenes, water vapor, oxygen, low-boiling hydrocarbons, possibly carbon oxides and possibly inert gases;
D) feeding of the gas stream c2 into an absorption zone and separation of incondensable and low-boiling gas constituents comprising oxygen, low-boiling hydrocarbons, possibly carbon oxides and possibly inert gases as gas stream d from the gas stream c2 by absorption of the C₄-hydrocarbons comprising butadiene and n-butenes in an absorption medium, giving an absorption medium stream d1 loaded with C₄-hydrocarbons and a recycle gas stream d2, and recirculation, optionally after a purge gas stream p has been separated off, of a substream d2m of the total recycle gas stream d2 into the reactor Rm,
wherein during the regeneration phase of the reactor Rm further reactors are in the production phase and the regeneration phase of the reactor Rm comprises the steps in the order i) to v):
i) reduction of the feed gas stream a1m comprising n-butenes and of the oxygen-comprising gas stream a2m and feeding of an inert gas stream a4m into the reactor Rm, with the total gas flow through the reactor Rm corresponding to from 55 to 75%, of the total gas flow through the reactor Rm during the production phase;
ii) further reduction of the feed gas stream a1m comprising n-butenes down to 0, reduction of the recycle gas substream d2m and increase of the inert gas stream a4m until the oxygen content in the reactor Rm is from 2 to 3% by volume, with the total gas flow through the reactor Rm corresponding to from 55 to 75% of the total gas flow through the reactor Rm during the production phase;
iii) regeneration of the catalyst by burning off carbon-comprising deposits at an oxygen content of from 2 to 3% by volume;
iv) reduction of the inert gas stream a4m and increase of the recycle gas substream d2m until the oxygen content in the reactor Rm is from 4 to 10% by volume, with the total gas flow through the reactor Rm corresponding to from 60 to 120% of the total gas flow during the production phase;
v) reoxidation of the catalyst at an oxygen content of from 4 to 10% by volume.

2. The process according to claim 1, wherein the oxygen:n-butenes ratio during step i) is from 0.7 to 1.0 times the ratio in the production phase.

3. The process according to claim 1, wherein the oxygen content in the reactor Rm is decreased to from 3 to 7% by volume during step i).

4. The process according to claim 1, wherein the gas stream a1m comprising n-butenes is reduced during step i) to from 25 to 50% of the gas flow during the production phase.

5. The process according to claim 1, wherein, during step ii), the gas stream a1m comprising n-butenes is reduced to from 12.5 to 25% of the gas flow during the production phase and the gas substream d2m is correspondingly reduced so that the oxygen:n-butenes ratio is from 0.7 to 1.0 times the ratio in the production phase, and the gas stream a1m comprising n-butenes is subsequently reduced to 0.

6. The process according to claim 1, wherein the step iii) is carried out over a time of from 5 to 60 minutes.

7. The process according to claim 1, wherein the step v) is carried out over a time of from 12 to 96 hours.

8. The process according to claim 1, wherein the ratio of oxygen to hydrocarbons in the production phase at an n-butenes content of from 50 to 100% by volume in the feed gas stream a1 is from 1.3:1 to 1.65:1.

9. The process according to claim 1, wherein the pressure in the reactor Rm during the production and regeneration phase is from 1 to 5 bar.

10. The process according to claim 1, wherein the regeneration phase is followed by a start-up phase comprising the steps vi) to viii):
vi) optionally additional feeding of a stream of steam a3m into the dehydrogenation zone;
vii) additional feeding-in of the feed gas stream a1m comprising butenes as a lower volume flow than in the production phase and increasing of this volume flow until at least 50% of the volume flow of the feed gas stream a1m in the production phase is reached, with the total gas flow through the reactor Rm corresponding to not more than 120% of the total gas flow during the production phase;
viii) additional feeding-in, when at least 50% of the volume flow of the feed gas stream a1m comprising butenes in the production phase is reached, of the oxygen-comprising stream a2m at a lower volume flow than in the production phase, and increasing of the volume flows of the gas streams a1m and a2m until the volume flows in the production phase have been reached, with the total gas flow through the dehydrogenation zone corresponding to not more than 120% on the total gas flow during the production phase.

11. The process according to claim 10, wherein the total gas stream comprising the streams a1m, a2m, d2m and optionally a3m through the dehydrogenation zone during steps (vi), (vii) and (viii) remains essentially constant and corresponds to from 90 to 110% by volume of the total gas flow through the dehydrogenation zone during the production phase.

12. The process according to either claim 10, wherein the amount of steam in the dehydrogenation zone during steps vi), vii) and viii) is from 0.5 to 10% by volume.

13. The process according to claim 10, wherein the pressure in the reactor Rm during the start-up phase is from 1 to 5 bar.

14. The process according to claim 1, wherein step D) comprises the steps Da) and Db):
Da) separation of incondensable and low-boiling gas constituents comprising oxygen, low-boiling hydrocarbons, possibly carbon oxides and possibly inert gases as gas stream d2 from the gas stream c2 by absorption of the C₄-hydrocarbons comprising butadiene and n-butenes in an absorption medium, giving an absorption medium stream d1 loaded with C₄-hydrocarbons and recycle gas stream d2, and
Db) subsequent desorption of the C₄-hydrocarbons from the loaded absorption medium stream d1, giving a C₄ product gas stream d3.

15. The process according to claim 14 having the additional steps:
E) separation of the C₄ product stream d3 by extractive distillation using a solvent which is selective for butadiene into a stream e1 comprising butadiene and the selective solvent and a stream e2 comprising n-butenes;
F) distillation of the stream f2 comprising butadiene and the selective solvent into a stream g1 consisting essentially of the selective solvent and a stream g2 comprising butadiene.
